(19) United States
(12) Patent Application Publication
(10) Pub. No.: US 2024/0425556 A1
(43) Pub. Date: Dec. 26, 2024

(54) MYOPEPTIDES AND METHODS OF USE IN TREATING HEART FAILURE

(71) Applicants: University of Cincinnati, Cincinnati, OH (US); Amgen Inc., Thousand Oaks, CA (US)

(72) Inventors: Sakthivel Sadayappan, Sr., Blue Ash, OH (US); Rohit Singh, Cincinnati, OH (US); Brandon Lee Ason, Pacifica, CA (US)

(73) Assignees: University of Cincinnati, Cincinnati, OH (US); Amgen Inc., Thousand Oaks, CA (US)

(21) Appl. No.: 18/685,037

(22) PCT Filed: Aug. 19, 2022
(86) PCT No.: PCT/US2022/075212

§ 371 Date: Feb. 20, 2024

Related U.S. Application Data
(60) Provisional application No. 63/235,439, filed on Aug. 20, 2021.

(51) Int. Cl. C07K 14/47; A61K 9/00; A61K 38/00; A61P 9/04

(52) U.S. Cl. CPC C07K 14/4716; A61K 9/0019; A61P 9/04; A61K 38/00; C07K 2319/33

(57) ABSTRACT

Myopeptides are provided, which include a myosin S2 fragment having at least 90% sequence identity to an amino acid sequence consisting of VKEMTERLEDEEEMNAELTAKK (SEQ ID NO: 1); and, optionally, a cardiac-homing peptide tag. Also provided are pharmaceutical compositions and methods of use of the myopeptides and pharmaceutical compositions in the treatment of heart failure.

### CROSS-REFERENCE TO RELATED APPLICATIONS

This application claims priority to U.S. Provisional Application Ser. No. 63/235,439,filed Aug. 20, 2021, the entire contents of which are incorporated herein by reference.

### SEQUENCE LISTING

A computer-readable form (CRF) sequence listing having file name CIN0349WO.xml. created on August 17, 2022 (22,000 bytes), is incorporated herein by reference. The amino acid sequences listed in the accompanying sequence listing are shown using standard abbreviations as defined in 37 C.F.R. 1.822. wherein:

SEQ ID NO: 1 corresponds to an amino acid sequence encoding for a portion of mouse myosin S2;

SEQ ID NO: 2 corresponds to an amino acid sequence encoding for a portion of human myosin S2;

SEQ ID NO: 3 corresponds to an amino acid sequence encoding for mouse myosin S2;

SEQ ID NO: 4 corresponds to an amino acid sequence encoding for human myosin S2;

SEQ ID NOs: 5-12 correspond to various amino acid subsequences of mouse myosin S2and control sequences;

SEQ ID NOs: 13-20 correspond to various myopeptides according to the present disclosure; and

SEQ ID NOs: 21-24 correspond to various cardiac-homing peptide tags according to the present disclosure.

### TECHNICAL FIELD

The present disclosure relates to the field of cardiology. More precisely, embodiments disclosed herein relate to myopeptides and their use in the treatment of heart failure.

### BACKGROUND

Heart failure is a major disease affecting the human population. Two types of heart failure include failure of the heart to pump enough blood into circulation, or systolic heart failure with reduced ejection fraction (HFrEF); and diastolic heart failure, or heart failure with preserved ejection fraction (HFpEF). In the case of HFpEF, the heart is unable to fill itself with a normal amount of blood, even though displaying normal ejection fraction greater than or equal to 70%. In HFpEF, the volume of blood reduces through each contraction, thus leading to development of a disease etiology, often later in life. HFrEF typically arises from coronary artery disease. hypertension, hypercholesterolemia, diabetes, smoking, obesity, heart valve disease, familial history of heart disease, dilated cardiomyopathy, or exposure to cardio toxic agents, such as alcohol, cocaine, amphetamines, cancer treatment, and radiation. Such secondary factors result in weakened left ventricle muscle unable to sustain regular ejection fraction for the human body.

HFpEF mostly occurs in the elderly, but may also arise due to hypertension, diabetes or genetic mutations leading to left ventricular hypertrophic cardiomyopathy. In HFpEF. the left ventricle becomes rigid, thickened and bulky so that cardiac muscle cannot relax. Current therapies for HFpEF and HFrEF involve the use of diuretics, vasodilators, beta blockers, inotropes, aldosterone antagonists, angiotensin receptor blockers and digoxin. Heart failure medicines that improve patient outcomes treat comorbidities such as blood pressure, thereby reducing the load or stress placed on the heart. Many heart failure medicines are not recommended for long-term use, as they result in diminished prognoses even though they improve heart failure symptoms. Currently, no heart failure therapeutics directly target cardiac muscle.

Diastolic heart failure with preserved ejection fraction (HFpEF) comprises 70% of all heart failure cases in the world, but a specific cure does not currently exist. In HFpEF. cardiac myosin binding protein-C (cMyBP-C) is highly dephosphorylated, which increases its interaction with myosin motors in the heart muscle (see FIG. 2C). The increased interaction between cMyBP-C and myosin promotes the inactivation of myosin motors for contraction in heart muscles during the development of HFpEF as well as systolic heart failure with reduced ejection fraction (HFrEF). Thus, reducing the interaction between cMyBP-C and myosin represents a potential target for preventing the development of HFpEF and treating HFrEF (see FIG. 2D).

A need exists for compositions and methods for the targeted treatment of different forms of heart failure.

### SUMMARY

Accordingly, provided herein are myopeptides that compete for binding of cMyBP-C. thereby reducing its interaction with myosin and improving heart contractility. Reducing the interaction between cMyBP-C and myosin promotes greater contractility of the heart and a net increase in the maximal force produced by failing heart muscle, which serves to prevent and treat various forms of heart failure.

In one embodiment, a myopeptide is provided, comprising (a) a myosin S2 fragment comprising a sequence having at least 90% sequence identity to an amino acid sequence consisting of VKEMTERLEDEEEMNAELTAKK (SEQ ID NO: 1); and (b) a cardiac-homing peptide tag.

In another embodiment, a pharmaceutical composition is provided, comprising a myopeptide according to the present disclosure and one or more pharmaceutically-acceptable excipients.

In another embodiment, a method of treating heart failure in a subject in need thereof is provided, the method comprising administering to the subject an effective amount of a myopeptide comprising a myosin S2 fragment having at least 90% sequence identity to an amino acid sequence consisting of VKEMTERLEDEEEMNAELTAKK (SEQ ID NO: 1).

These and other objects, features, embodiments, and advantages will become apparent to those of ordinary skill in the art from a reading of the following detailed description and the appended claims.

### BRIEF DESCRIPTION OF DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1 is a schematic diagram depicting the features of HFpEF (middle panel) and HFrEF (right panel) heart failure compared to a normal heart (left panel).

FIG. 2A is a schematic illustrating the coiled structure of myosin, with the C-terminal end at the left and the N-terminal end at the right.

FIG. 2B is a schematic illustrating cMyBP-C protein and its functional domains.

FIG. 2C is a schematic illustrating structural changes that take place in cardiac tissue upon phosphorylation of cMyBP-C protein. The schematic shows the position of cMyBP-C in a sarcomere where cMyBP-C is anchored on muscle thick filament at its carboxyl terminal end. while the amino-terminal end can interact with actin and myosin.

FIG. 2D is a schematic illustrating the structural changes that take place in cardiac muscle upon treatment with a myopeptide according to embodiments of the present disclosure.

FIG. 3A is a graph showing results from a solid phase binding assay for competitive binding between various myopeptide sequences and S2126-C0-C2, specifically for experimental peptides Amg 23003-23006 (SEQ ID NOs: 5-8, respectively). Amg 23005 and Amg 23006displayed a decrease in relative fluorescence with increasing concentration, showing their ability to compete for S2¹²⁶-C0-C2 binding and thus dropping the relative fluorescence to 0 at 20 μM. stating a stoichiometry of 1 with C0-C2 protein.

FIG. 3B is a graph showing results from a solid phase binding assay for competitive binding between various myopeptide sequences and S2¹²⁶-C0-C2, specifically for experimental peptides Amg 27117-27120 (SEQ ID NOs: 1, 10-12, respectively). As depicted. Amg 27119 is competitive for S2¹²⁶-C0-C2 binding, as shown by the drop in fluorescence to 0 at concentrations above 15 μM.

FIG. 4A depicts graphs showing maximal force produced by skinned papillary muscle fibers versus calcium concentration for experimental myopeptides Amg 23003 (SEQ ID NO: 5,top left panel); Amg 23004 (SEQ ID NO: 6. top right panel); Amg 23005 (SEQ ID NO: 7, bottom left panel); and Amg 23006 (SEQ ID NO: 8, bottom right panel).

FIG. 4B depicts graphs showing maximal force produced by skinned papillary muscle fibers versus calcium concentration for experimental myopeptides Amg 27117 (SEQ ID NO: 1,top left panel); Amg 27118 (SEQ ID NO: 10, top right panel); Amg 27119 (SEQ ID NO: 11,bottom left panel), and Amg 27120 (SEQ ID NO: 12, bottom right panel).

FIG. 5A depicts graphs showing the rates of force redevelopment in skinned papillary fibers for experimental myopeptide sequences Amg 23003 (SEQ ID NO: 5, top left panel), Amg 23004 (SEQ ID NO: 6, top right panel); Amg 23005 (SEQ ID NO: 7. bottom left panel): and Amg 23006 (SEQ ID NO: 8, bottom right panel).

FIG. 5B depicts graphs showing the rates of force redevelopment in skinned papillary fibers for experimental myopeptide sequences Amg 27117 (SEQ ID NO: 1, top left panel); Amg 27118 (SEQ ID NO: 10, top right panel); Amg 27119 (SEQ ID NO: 11, bottom left panel); and Amg 27120 (SEQ ID NO: 12, bottom right panel).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to achieve the disclosed subject matter.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including.” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used herein the singular forms “a.” “an” and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure.

### Myopeptides

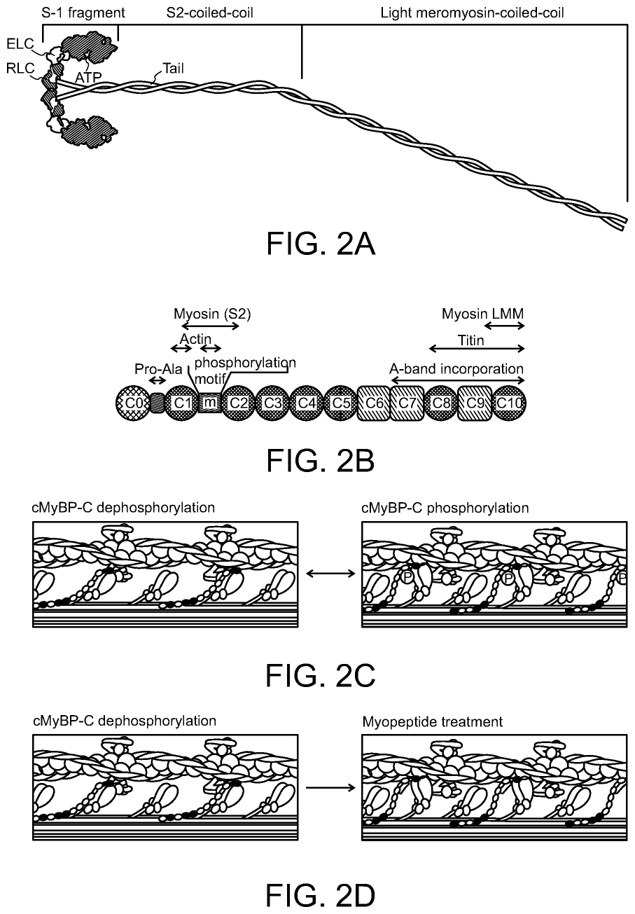

Myosin is a major motor protein in the heart muscle comprised of approximately 1900amino acids that drives the contraction and relaxation of heart muscle. Myosin is comprised of two components: heavy meromyosin and light meromyosin. Heavy meromyosin is further divided into subunits, namely myosin sub-fragment 1 (S1) and myosin sub-fragment 2 (S2). S1 is comprised of approximately 810 amino acids and acts as the functional end of myosin. hydrolyzing adenosine triphosphate (ATP), thereby resulting in the contraction of heart muscle. S2 is comprised of approximately 600 amino acids and is an alpha-helical coiled-coil protein. which acts as a linker between S1 and light meromyosin. Myosin S2 binds cMyBP-C. As seen in FIGS. 2A-2D, myosin S2 serves as a binding site for cMyBP-C when cMyBP-C is in its dephosphorylated state.

Cardiac Myosin Binding Protein-C (cMyBP-C) is a thick filament accessory protein in the sarcomere and an important regulator of muscle contraction via phosphorylation at the molecular level. Phosphorylated cMyBP-C allows for the activation of contraction in muscles, but when dephosphorylated, it inactivates contraction by binding to myosin at its sub-fragment 2 region (S2). In HFpEF. the phosphorylation level of cMyBP-C is significantly diminished. The reduced phosphorylation of cMyBP-C in HFpEF has been observed in patients of HF owing to cardiomyopathy, hypertension, diabetes and aging. Consequently, dephosphorylated cMyBP-C subverts normal heart muscle function by acting as brake against the normal motor function of myosin.

cMyBP-C is a protein comprised of 1273 amino acids and 8 immunoglobulin (Ig)-like domains: C0-C5, C8, and C10. cMyBP-C comprises 3 fibronectin-like domains: C6, C7, and C9.cMyBP-C also consists comprises a short stretch of amino acids between the C1 and C2 domains called the m-motif (see FIG. 2B). The m-motif comprises three serine residues at positions 273, 282, and 302, the phosphorylation of which governs the activity of cardiac muscle. When phosphorylated, cMyBP-C binds to actin thin filaments in heart muscle, but when cMyBP-C undergoes dephosphorylation of the noted serine residues, it binds to myosin S2 (as seen in FIGS. 2C and 2D).

It has been established that the initial N-terminal domains of cMyBP-C. i.e., C0-C2, can sufficiently bind myosin S2, as well as a proximal sub-fragment 2 region consisting of 126 amino acids. The 126 amino acid fragment of mouse S2 (SEQ ID NO: 3) and corresponding 129 amino acid fragment of human S2 (SEQ ID NO: 4) are highly conserved between the genes MHY6 (alpha myosin heavy chain) and MHY7 (beta myosin heavy chain). It has previously been shown that the proximal sub-fragment 2 region of 126 amino acids (S2126) binds the C0-C2 domains of cMyBP-C, whereas S2 fragments missing the proximal 126 residues cannot bind the C0-C2 protein domains.

As used herein. the term “myopeptide” refers to a peptide that interrupts the protein-protein interaction of M2 and cMyBP-C. In embodiments, a myopeptide according to the present disclosure comprises a fragment or portion of myosin S2 (SEQ ID NOs: 4 or 5). In embodiments, a myopeptide comprises a sequence having at least 90% sequence identity to an amino acid sequence consisting of SEQ ID NO: 1 or SEQ ID NO: 2. In embodiments, a myopeptide comprises a sequence having at least 95%, at least 96%, at least 97%, at least 98%, or at least 99% sequence identity to SEQ ID NO: 1 or SEQ ID NO: 2. In embodiments, the myopeptide binds to cardiac myosin binding protein-C. In embodiments, the myopeptide competitively binds the cMyBP-C protein, thereby preventing other species from binding.

In embodiments, the myopeptide comprises fewer than 50, 45, 40, 35, 30, or 25 amino acids. In specific embodiments, the myopeptide is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length, or any range therebetween. In specific embodiments, the myosin S2 fragment is at least 20, at least 21, at least 22, at least 23, at least 24, or at least 25 amino acids in length.

As used herein, the term “myosin S2 fragment” refers to a functional portion of the myosin S2 amino acid sequence that is capable of competitively binding cardiac myosin binding protein- C. In embodiments, the myosin S2 fragment of the present myopeptides comprises an amino acid sequence selected from SEQ ID NO: 1 or SEQ ID NO: 2, or functional equivalents thereof. In embodiments, the myosin S2 fragment comprises a sequence having at least 90%, at least 95%, at least 96%, at least 97%, at least 98%, or at least 99% sequence identity to SEQ ID NO: 1 or SEQ ID NO: 2.

As used herein “C0-C2” refers to the C0, C1, and C2 domains of cardiac myosin binding protein-C. In embodiments, the C0-C2 domains of the cMyBP-C protein binds to the myosin S2 fragment.

As used herein, the term “subject” generally refers to a living being (e.g., an animal or human). In some embodiments, a subject described herein may be a patient to be treated therapeutically or may be employed as a means for generating tools for research, diagnostic, and/or therapeutic purposes. In a specific embodiment, the subject is selected from a mouse, rat, rabbit, chicken, pig, monkey, and human. In a more specific embodiment, the subject is a human.

In embodiments, the myopeptide further comprises a cardiac-homing peptide tag. The term “cardiac-homing peptide tag” refers to a short peptide sequence of about 5 to about 15 amino acids that can be conjugated to a myopeptide to target the myopeptide for selective homing to cardiac tissue. As used herein, the term “selectively home.” when used in reference to a cardiac-homing peptide, means a peptide that selectively binds to normal cardiac tissue or pathologic cardiac tissue, such as ischemic tissue, in preference to other non-cardiac tissues of the body. In general, a cardiac-homing peptide is characterized by at least a two-fold greater selective binding of the peptide to cardiac tissue as compared to a control tissue, such as the brain.

In some embodiments, the cardiac-homing peptide tag may be added to the C-terminus, the N-terminus, or both the C-and N-terminus of a myopeptide as disclosed herein, such as SEQ ID NO: 1 or SEQ ID NO: 2. In embodiments, the cardiac-homing peptide delivers the myopeptide to the cardiac muscle of a subject.

In embodiments, the cardiac-homing peptide tag comprises an amino acid sequence having at least 90%, at least 95%, at least 96%, at least 97%, at least 98%, or at least 99% sequence identity to SEQ ID NO: 21, SEQ ID NO: 22, SEQ ID NO: 23, or SEQ ID NO: 24. In a specific embodiment, the cardiac-homing peptide tag sequence comprises SEQ ID NO: 21, SEQ ID NO: 22, SEQ ID NO: 23, or SEQ ID NO: 24. Exemplary suitable cardiac-homing peptides are disclosed. for example, in U.S. Pat. No. 6,303,573, issued Oct. 15, 2001. to Ruoslahti, incorporated herein by reference.

In embodiments, a cardiac-homing peptide tag is conjugated to the N-terminus of a myopeptide to produce a tagged myopeptide. For example, a tagged myopeptide may include a cardiac-homing peptide according to SEQ ID NO: 21, SEQ ID NO: 22, SEQ ID NO: 23, or SEQ ID NO: 24 added to the N-terminus of a myopeptide according to SEQ ID NO: 1 or SEQ ID NO: 2.

For example, in a specific embodiment, the cardiac-homing tag of SEQ ID NO: 21 is added to the N-terminus of SEQ ID NO: 1 to provide the myopeptide of SEQ ID NO: 13. In another embodiment, the cardiac-homing tag of SEQ ID NO: 22 is added to the N-terminus of SEQ ID NO: 1 to provide the myopeptide of SEQ ID NO: 15. In another embodiment, the cardiac-homing peptide of SEQ ID NO: 23 is added to the N-terminus of SEQ ID NO: 1 to provide the myopeptide of SEQ ID NO: 17. In another embodiment, the cardiac-homing peptide of SEQ ID NO: 24 is added to the N-terminus of SEQ ID NO: 1 to provide the myopeptide of SEQ ID NO: 19.

In embodiments, the cardiac-homing tag is added to the C-terminus of a myopeptide to provide a tagged myopeptide. For example, the cardiac-homing peptide according to SEQ ID NO: 21, SEQ ID NO: 22, SEQ ID NO: 23, or SEQ ID NO: 24 may be added to the C-terminus of a myopeptide according to SEQ ID NO: 1 or SEQ ID NO: 2. In a specific embodiment, the cardiac-homing tag of SEQ ID NO: 21 is added to the C-terminus of SEQ ID NO: 1 to provide the myopeptide of SEQ ID NO: 14. In another embodiment, the cardiac-homing tag of SEQ ID NO: 22 is added to the C-terminus of SEQ ID NO: 1 to provide the myopeptide of SEQ ID NO: 16. In another embodiment, the cardiac-homing tag of SEQ ID NO: 23 is added to the C-terminus of SEQ ID NO: 1 to provide the myopeptide of SEQ ID NO: 18. In another embodiment, the cardiac-homing tag of SEQ ID NO: 24 is added to the C-terminus of SEQ ID NO: 1 to provide the myopeptide of SEQ ID NO: 20.

In specific embodiments, myopeptides of the present disclosure include myopeptides comprising a sequence having at least 90%, at least 95%, at least 96%, at last 97%, at least 98%. at least 99%, or 100% sequence identity with SEQ ID NO: 1, SEQ ID NO: 2, SEQ ID NO: 13,SEQ ID NO: 14, SEQ ID NO: 15, SEQ ID NO: 16, SEQ ID NO: 17, SEQ ID NO: 18, SEQ ID NO: 19, or SEQ ID NO: 20.

The myopeptides and cardiac-homing tags set forth herein may include modifications to their respective amino acid sequences, so long as the binding and cardiac-homing functions thereof are maintained. A functionally equivalent modification can be, for example, one, two, or more amino acid additions or substitutions relative to the reference peptide sequence. In embodiments, a conservative substitution is the replacement of a first amino acid with a second amino acid having similar biochemical properties. For example, a first nonpolar amino acid can be conservatively substituted with a second (non-identical) nonpolar amino acid such as alanine, valine, leucine, isoleucine, proline, methionine, phenylalanine, or tryptophan. Similarly, a first uncharged amino acid can be conservatively substituted with a second uncharged polar amino acid such as glycine, serine, threonine, cysteine, tyrosine, asparagine, or glutamine. In addition, a first negatively charged amino acid can be conservatively substituted with a second negatively charged amino acid such as aspartic acid or glutamic acid. In the same way, a first positively charged amino acid can be conservatively substituted with a second positively charged amino acid such as lysine, arginine or histidine. Non-conservative substitutions, which also are encompassed by the disclosure, involve the replacement of an amino acid of one class, for example, a nonpolar residue. with an amino acid of a second class, for example, a polar or charged residue.

As used herein, “maximal force” refers to the maximum amount of force produced by cardiac muscle fibers. In embodiments, the force generated by the cardiac muscle fibers is used to improve contractility in the heart.

In embodiments, the maximum force generated by cardiac muscle fibers when contacted by a myopeptide according to the present disclosure is increased by about 1% to 30%, 1% to 25%, 1% to 20%, 1% to 15%, 1% to 10%, 5% to 30%, 5% to 25%, 5% to 20%, 5% to 15%, 5% to 10%, 10% to 30%, 10% to 25%, 10% to 20%, 10% to 15%, 15% to 30%, 15% to 25%, 15% to 20%, 20% to 30%, 20% to 25%, 25% to 30%, or 30%. In a specific embodiment, the maximum force is increased by about 30%.

As used throughout this disclosure, rate of actin and myosin cross-bridge formation is designated as k_{tr,}. In embodiments, a myopeptides of the present disclosure increase the kₜᵣ by up to about 2.5 times the rate of a control. In a specific embodiment, a myopeptide as described herein increases the kₜᵣ by 2.5 times the rate of the control. In another specific embodiment, the myopeptide increases the kₜᵣ by 1.5 times the control. In embodiments. 10 μM of the myopeptide is sufficient to significantly increase the kₜᵣ. In other embodiments. 20 μM of the myopeptide is sufficient to significantly increase the kₜᵣ.

Systolic heart failure with reduced ejection fraction, or HFrEF, is a cardiac condition defined by eccentric left ventricular hypertrophy, a reduced ejection fraction of less than 40%, and systolic dysfunction.

Diastolic heart failure with preserved ejection fraction, or HFpEF, is a complex cardiac syndrome with an ejection fraction of greater than 50%, concentric left ventricular hypertrophy. and diastolic dysfunction. The skilled artisan will appreciate that HFpEF is recognized as an early stage preceding development of HFrEF.

### Pharmaceutical Compositions

In embodiments, a pharmaceutical composition is provided, the composition comprising an effective amount of a myopeptide according to any of the embodiments disclosed herein and a pharmaceutically-acceptable excipient.

The pharmaceutically acceptable excipient, or carrier, must be “acceptable” in the sense of being compatible with the other ingredients of the pharmaceutical composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for parenteral administration. In a specific embodiment, the compositions disclosed herein are suitable for intramuscular administration, although other specific means of parenteral administration are also viable (such as, for example, intravenous, infusion, intra-arterial, or subcutaneous administration). The compositions may be prepared by any methods well known in the art of pharmacy. for example. using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given pharmaceutical composition.

For parenteral administration, suitable compositions include aqueous and non-aqueous sterile suspensions for intramuscular and/or intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, the pharmaceutical composition may be formulated for injection. In other embodiments, the pharmaceutical composition may be formulated for infusion. In a specific embodiment, the pharmaceutical composition is formulated for an intramuscular injection, for example, to the cardiac muscle.

The term “effective amount,” as used herein, refers to the amount of a composition that is sufficient to achieve a desired biological effect. Generally, the dosage needed to provide an effective amount of the composition will vary depending upon such factors as the subject's age. condition, sex, and other variables which can be adjusted by one of ordinary skill in the art. The compositions of the present disclosure can be administered by either single or multiple dosages of an effective amount. In a specific embodiment, the effective amount is an amount sufficient to elicit the competitive binding of the myopeptide to cMyBP-C in the cardiac muscle of a subject.

### Methods of Treating Heart Failure

In embodiments, a method of treating heart failure in a subject in need thereof is provided, the method comprising administering to the subject an effective amount of a myopeptide comprising a myosin S2 fragment having at least 90% sequence identity to an amino acid sequence consisting of VKEMTERLEDEEEMNAELTAKK (SEQ ID NO: 1) or VKEMNERLEDEEEMNAELTAKK (SEQ ID NO: 2). In specific embodiments, the myosin S2 fragment comprises a sequence having at least 95%, at least 96%, at least 97%, at least 98%, or at least 99% sequence identity to SEQ ID NO: 1 or SEQ ID NO: 2.

In embodiments, a method of treating heart failure in a subject in need thereof is provided. the method comprising administering to the subject an effective amount of a pharmaceutical composition comprising a myopeptide according to the present disclosure and a pharmaceutically-acceptable excipient.

The terms “treat,” “treatment,” and “treating,” as used herein, refer to a method of alleviating or abrogating a disease, disorder, and/or symptoms thereof. In a specific embodiment, the disease or disorder is heart failure. In a more specific embodiment, the disease to be treated is heart failure with preserved ejection fraction (HFpEF) or heart failure with reduced ejection fraction (HFrEF).

In embodiments, the method further comprises administering to the subject a second therapeutic agent. In embodiments, the second therapeutic agent is an agent typically administered to treat the symptoms of heart failure. In embodiments, the second therapeutic agent is selected from the group consisting of diuretics, vasodilators, beta blockers, inotropes, aldosterone antagonists, angiotensin receptor blockers, and digoxin.

In embodiments, the myopeptide according to the present disclosure and the second active agent are co-administered. “Co-administered,” as used herein, refers to administration of the myopeptide and the second therapeutic agent such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the myopeptide and the second therapeutic agent may be administered concurrently or sequentially.

In embodiments, the methods described herein do not alter the calcium sensitivity of the subject.

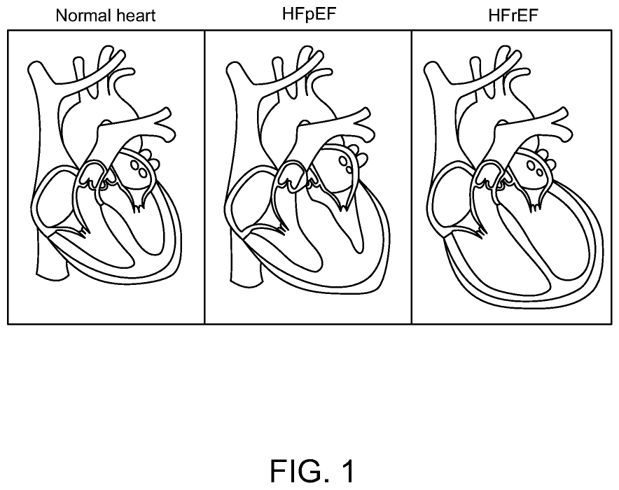

Referring to FIG. 1, a schematic diagram displaying the two forms of heart failure. preserved ejection fraction (HFpEF) and reduced ejection fraction (HFrEF) is depicted. A normal heart is depicted in the leftmost panel, a heart displaying HFpEF is depicted in the middle panel, and a heart displaying HFrEF is depicted in the rightmost panel. HFpEF is a complex syndrome presenting with an ejection fraction (EF) of greater than 50%, concentric left ventricular hypertrophy, and diastolic dysfunction. In contrast, HFrEF is defined by the presence of eccentric left ventricular hypertrophy, reduced EF of less than 40%, and systolic dysfunction. HFpEF is recognized as an early stage of HFrEF, providing motivation for its early prevention and treatment.

Referring to FIGS. 2A and 2B, schematics displaying the structure and function of myosin S2 and cMyBP-C are depicted. FIG. 2A shows a cartoon representation of the coiled structure of myosin, with different subunits labeled from the C-terminal region to the N-terminal region (i.e., from left to right). ELC refers to the essential light chai, and RLC refers to the regulatory light chain. FIG. 2B is a diagram of cardiac myosin binding protein-C and its different domains, comprising eight immunoglobulin domains, three fibronectin domains, an m-domain between the C1 and C2 domains, and a cardiac muscle-specific C0 domain with a proline-alanine linker. The parts of the sarcomere proteins binding to cMyBP-C are labelled. FIG. 2C displays the position of cMyBP-C in the sarcomere, where cMyBP-C is anchored on muscle thick filament at its C-terminal end, while the N-terminus can interact with actin and myosin. FIGS. 2C and 2D (left panels) display the serine residues at the m-domain of cMyBP-C. When dephosphorylated during HF and HFpEF, cMyBP-C remains bound to myosin S2. When phosphorylated or when a myopeptide of the present disclosure competitively binds (right panels), cMyBP-C binds to actin thin filaments, thereby freeing myosin S1 heads from S2 to bind actin filaments.

### EXAMPLES

The following examples are given by way of illustration and are in no way intended to limit the scope of the present disclosure.

### Example 1: Design of Peptide Sequences

The native mouse myosin S2 126 amino acid sequence to which cMyBP-C binds is PLLKSAETEKEMANMKEEFGRVKDALEKSEARRKELEEKMVSLLQEKNDLQLQVQAE QDNLNDAEERCDQLIKNKIQLEAKVKEMTERLEDEEEMNAELTAKKRKLEDECSELKK DIDDLELTLAK. (SEQ ID NO: 3).

The native amino acid sequence shares commonality among the MYH6 gene from mus musculus (UniProt ID-Q02566) amino acids 840-965, and among the MYH7 gene from homo sapiens (UniProt ID-P12883) amino acids 838-963.

The peptides from Amg 23003 to 23006 were designed from the initial 126 residues with some overlapping amino acid residues. For example, RKELEEKM was common between Amg 23003 and 23004, CDQLIKNK was common between Amg 23004 and Amg 23005 and VKEMTERLEDEEEMNAELTAKK (SEQ ID NO: 1) was common between Amg 23005 and Amg 23006. Amg 25030 served as scramble control. Based on experimental testing, Amg 23005and Amg 23006 successfully improved muscle function. Therefore, Amg 23006 was used as a basis to create Amg 27117-Amg 27120, in which Amg 27119 (SEQ ID NO: 1) was the common sequence between Amg 23005 and Amg 23006. Table 1 sets forth various experimental sequences and components thereof employed in the design of the disclosed myopeptides.

TABLE 1
Experimental Amino Acid Sequences

| Sequence | | | |
|---|---|---|---|
| Identifier | Description | Sequence (5′-3′) | Mol. Wt. |
| SEQ ID NO: 1 | Mouse | VKEMTERLEDEEEMNAELTAKK | 2623.91 |
| | Myosin S2 | | |
| | subset | | |
| | Amg 27119 | | |
| SEQ ID NO: 2 | Human | VKEMNERLEDEEEMNAELTAKK | |
| | Myosin S2 | | |
| | subset | | |
| SEQ ID NO: 3 | Mouse | PLLKSAETEKEMANMKEEFGRVKDALEKSEARRK | |
| | Myosin S2 | ELEEKMVSLLQEKNDLQLQVQAEQDNLNDAEERC | |
| | | DQLIKNKIQLEAKVKEMTERLEDEEEMNAELTAK | |
| | | KRKLEDECSELKKDIDDLELTLAK | |
| SEQ ID NO: 4 | Human | GSSPLLKSAEREKEMASMKEEFTRLKEALEKSEAR | |
| | Myosin S2 | RKELEEKMVSLLQEKNDLQLQVQAEQDNLADAEE | |
| | | RCDQLIKNKIQLEAKVKEMNERLEDEEEMNAELT | |
| | | AKKRKLEDECSELKRDIDDLELTLAK | |
| SEQ ID NO: 5 | Amg 23003 | PLLKSAETEKEMANMKEEFGRVKDALEKSEARRK | 4710.41 |
| | | ELEEKM | |
| SEQ ID NO: 6 | Amg 23004 | RKELEEKMVSLLQEKNDLQLQVQAEQDNLNDAEE | 5127.73 |
| | | RCDQLIKNK | |
| SEQ ID NO: 7 | Amg 23005 | CDQLIKNKIQLEAKVKEMTERLEDEEEMNAELTA | 4249.88 |
| | | KK | |
| SEQ ID NO: 8 | Amg 23006 | VKEMTERLEDEEEMNAELTAKKRKLEDECSELKK | 5310.99 |
| | | DIDDLELTLAK | |
| SEQ ID NO: 9 | Scramble | ETEKEMAMVSLLQAEERCDQDIDDLELTLAK | 3567.97 |
| | control | | |
| | Amg 25030 | | |
| SEQ ID NO: 10 | Amg 27117 | DEEEMNAELTAKKR | 1663.8 |
| SEQ ID NO: 11 | Amg 27118 | KLEDECSELKKDIDDLELTLAK | 2548.86 |
| SEQ ID NO: 12 | Amg 27120 | ETEKEMAMVSLLQAE | 1708.91 |

### Example 2: In-Vitro Solid Phase Binding Assay

Peptides of Table 1 were tested in vitro to determine whether they compete for binding in the interaction between S2¹²⁶ and the C0-C2 protein. In these experiments, recombinant S2¹²⁶ and C0-C2 protein with 6X his-tags were bacterially expressed in BL21 E. coli and then purified through Talon© metal affinity columns. A solid phase binding assay (SPBA) was then performed, whereby 20 μM of the C0-C2 protein was adsorbed onto wells of a 96-well microtiter plate. Then, 20 μM of S2¹²⁶, along with increasing concentrations of selected experimental peptides from Table 1, ranging from 0-20 μM, were added.

The following procedure identified the competitive nature of the tested experimental peptides from Table 1. If the experimental peptide did not compete for S2¹²⁶-C0-C2 binding. then the primary antibody (rabbit) specific to S2¹²⁶ recognized S2¹²⁶. The Alexa Fluor 568© secondary antibody (anti-rabbit) would then bind to the primary antibody to give a relative positive fluorescence of 1.0. Conversely, if the experimental peptide successfully competed with the S2¹²⁶-C0-C2 binding. then the S2¹²⁶ site would not be available for binding by the primary antibody. Therefore, the secondary antibody would also fail to bind, leading to a decrease in the relative fluorescence below 1.0. As a result, for a non-competing peptide, the relative fluorescence approaches 1.0, as observed by S2¹²⁶-C0-C2 binding. For a competing peptide, a decay in the relative fluorescence is observed. This SPBA assay was used to screen peptides for efficacy in disrupting the interaction between S2¹²⁶-C0-C2.

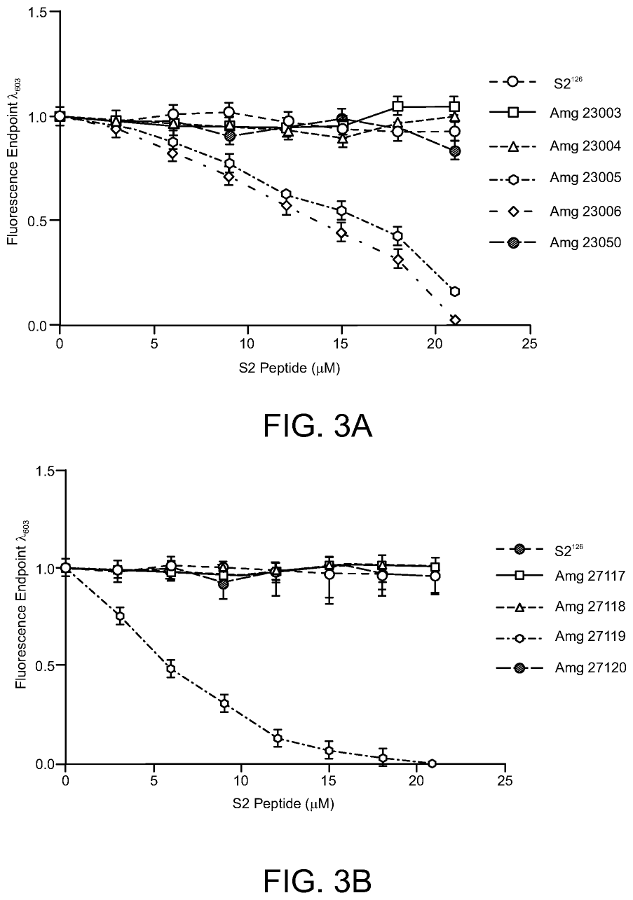

Results from the SPBA binding assay are shown in FIG. 3A. From the graph of fluorescence versus S2 peptide concentration (μM), it can be seen that the S2¹²⁶ peptide when bound to C0-C2 maintains a relative fluorescence near 1.0, thereby serving as a non-competing control. Amg 23003, Amg 23004, and Amg 23050 (the scramble control) all maintained a relative fluorescence near 1.0, and are thereby unable to compete for S2¹²⁶-C0-C2 binding, even at increasing concentrations of these peptides. However, Amg 23005 and Amg 23006 displayed a decay in relative fluorescence to 0 at 20 μM, showing a stoichiometry of 1 with the C0-C2 protein. Therefore, Amg 23005 and Amg 23006 were selected for further development as potential competitive experimental myopeptides.

Because Amg 23005 and Amg 23006 were the only two experimental peptides to competitively bind, and thereby improve muscle function, Amg 23006 was broken down further into Amg 27117-Amg 27120 in Table 1 for further testing. In FIG. 3B, Amg 27117-Amg 27120 were tested using an identical SPBA assay as described above. As shown in FIG. 3B, Amg 27117. 27118, and 27120 all retained a relative fluorescence of near 1.0. However, Amg 27119showed a decay in binding and a relative fluorescence of 0 above 15 μM, which meets the threshold as a competitive binding peptide for S2¹²⁶-C0-C2.

Taken together, the results of FIGS. 3A and 3B show that Amg 23005, 23006, and 27119all effectively compete for S2¹²⁶-C0-C2 binding.

### Example 3: Isothermal Titrating Calorimetry (ITC)

Isothermal titrating calorimetry (ITC) was used to validate and verify the results from the SPBA assays of Example 2. In ITC experiments, the heat of binding between two proteins is measured by titrating one protein with the other protein and then measuring the heat released or absorbed through a reference fluid with a constant temperature. By titrating 300 μM of S2¹²⁶ to 20 μM of C0-C2 protein, the stoichiometry (η) of 0.98 and dissociation constant (k_{d}) of 4.7 μM for S2¹²⁶-C0-C2 binding were determined.

Using the above stoichiometry and dissociation constant values as controls, all of the experimental peptides were titrated at 300 μM with 20 μM of C0-C2 protein and compared with the above S2¹²⁶ control. Peptides that displayed similar η, k_{d}, and heat of binding similar to S2¹²⁶-C0-C2 were selected for further testing in muscle fibers.

### Example 4: Effect on Contractility in Heart Muscle

The ability of the experimental peptides to increase or otherwise affect contractility in the heart muscle system was tested using skinned papillary fibers from mouse hearts. In this ex vivo experiment, cardiac papillary fibers from mouse hearts were isolated and skinned overnight in 1% Triton X-100 low calcium buffer supplemented with ATP, creatine phosphate, and magnesium chloride. Because skinned muscle fibers are devoid of sarcolemma, nuclei, and sarcoplasmic reticulum, the fibers can only contract if supplemented with calcium, magnesium chloride, ATP, and creatine phosphate. This assay allows for the opportunity to screen for compounds or molecules that affect the contractility of the muscle.

Skinned papillary muscle fibers were cycled through buffers containing a constant concentration magnesium chloride, ATP, creatine phosphate, but increasing concentrations of calcium. As the muscle fiber goes from buffers containing low concentrations of calcium to buffers containing high concentrations of calcium, the force developed increases with increasing concentration. Thus, plotting the force produced versus calcium concentration provides a sigmoidal curve, yielding the maximal force produced (Fₘₐₓ) and the midpoint calcium concentration where the force produced is exactly half of the force produced at high calcium concentration. This is useful for measuring the calcium sensitivity (pCa₅₀) of the muscle fibers.

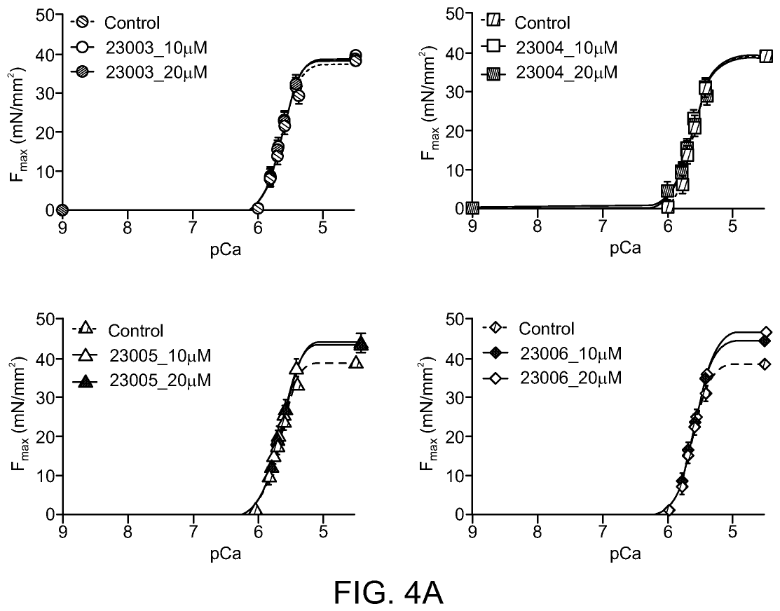

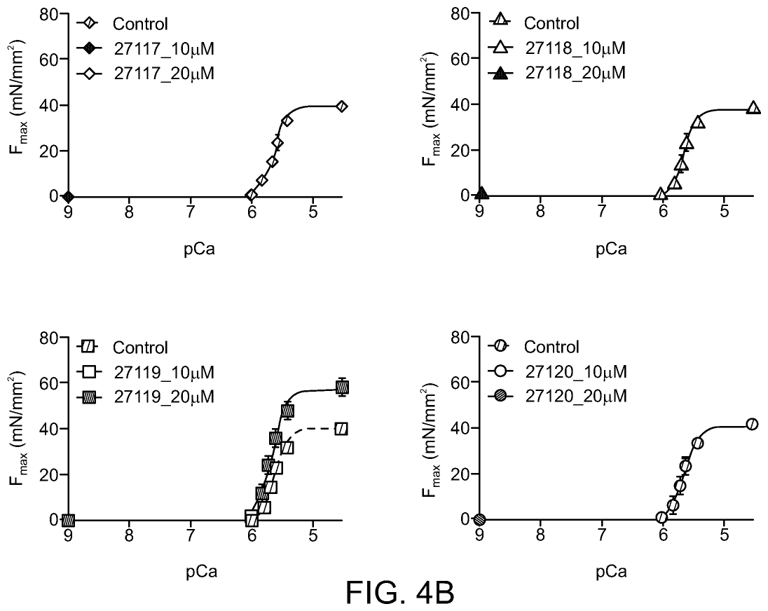

In the panels of FIG. 4A, the Fₘₐₓ (mN/mm²) is plotted against increasing calcium concentrations (from pCa 9.0 to pCa 5.0) for peptides Amg 23003-23006 at concentrations of 10 μM and 20 μM, and a control for comparison of each. Results show that Amg 23005 and Amg 23006 increased the maximal force produced by the fibers, by 13%, particularly at higher calcium concentrations (pCa 4.5).

In the panels of FIG. 4B, the same assay was carried out with peptides Amg 27117-27120. Results showed that Amg 27119 peptide increased the maximal force by about 30%, particularly at higher calcium concentrations of both 10 μM and 20 μM.

### Example 5: Measurement of Rate of Force Redevelopment

To calculate the rate of force redevelopment or actin-myosin cross-bridge formation (kₜᵣ) in cardiac muscle fibers, the fibers were allowed to reach steady state force in submaximal calcium concentration, and then the fiber was slackened by 2% of its length for 20 milliseconds. The slackening was immediately followed by rapid stretching of the fiber to its original length. The force is dropped when slackened, but begins to redevelop when the fiber is stretched, thus allowing an estimation of the time required for the fiber to develop force, or how quickly actin-myosin cross-bridges are formed.

The experiment and measurement of Fₘₐₓ, pCa₅₀, and kₜᵣ was performed for a muscle in the absence of the experimental peptide and then in the presence of 10 μM and 20 μM of the experimental peptide.

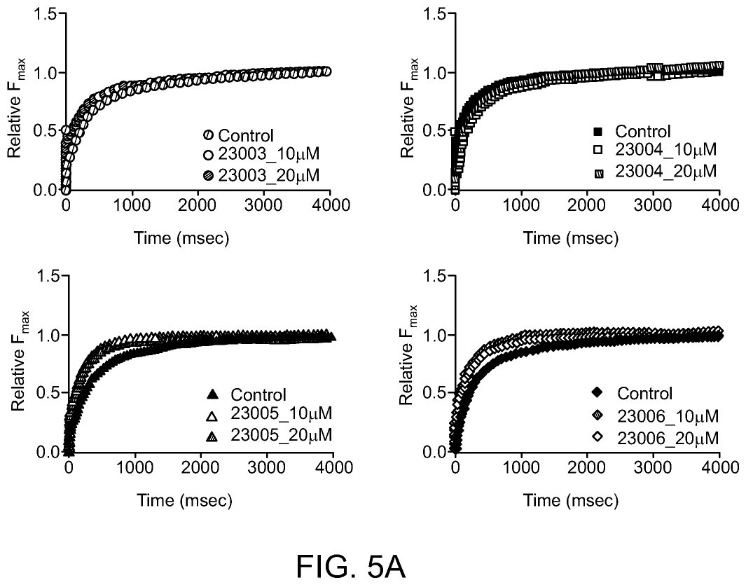

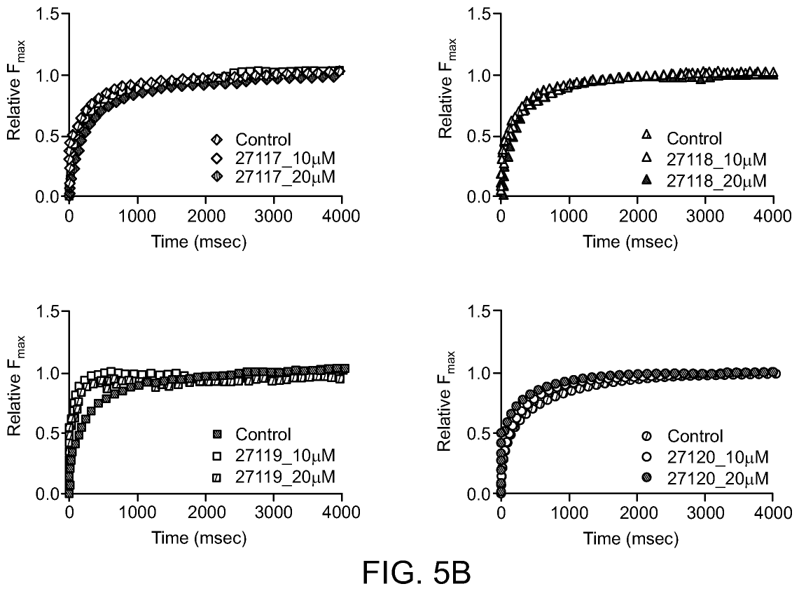

Results are shown in FIG. 5A and FIG. 5B. The rapid slack and stretch protocol was conducted in the presence of myopeptides Amg 23003-23006 at a calcium concentration of pCa 5.7. Results showed that experimental peptides Amg 23005 and 23006 increase the kₜᵣ by 1.5 fold at pCa 5.7, compared to the control. As shown in FIG. 5B, Amg 27119 also increased the kₜᵣ at pCa 5.7, by 2.5 fold compared to the control.

### Example 6: Tagging Peptide With Heart Homing Sequence

Amg 27119 was determined to be highly effective in improving contractile function in cardiac muscle through competitively binding C0-C2 of cMyBP-C. Advantageously, the amino acid sequence of Amg 27119 is also small enough to be effectively tagged with a cardiac-homing peptide. Cardiac-homing peptide tag sequences and myopeptides comprising the same are set forth in Table 2, wherein cardiac-homing tags are underlined within the myopeptide sequence.

TABLE 2
Exemplary myopeptides comprising cardiac-homing peptide tags

| Sequence | | | |
|---|---|---|---|
| Identifier | Description | Sequence (5′-3′) | Mol. Wt. |
| SEQ ID NO: 13 | Amg 27119 IN | GGGVFWQVKEMTERLEDEEEMNAELTAKK | 3355.74 |
| SEQ ID NO: 14 | Amg 27119 1C | VKEMTERLEDEEEMNAELTAKKGGGVFWQ | 3355.74 |
| SEQ ID NO: 15 | Amg 27119 2N | HGRVRPHVKEMTERLEDEEEMNAELTAKK | 3463.89 |
| SEQ ID NO: 16 | Amg 27119 2C | VKEMTERLEDEEEMNAELTAKKHGRVRPH | 3463.89 |
| SEQ ID NO: 17 | Amg 27119 3N | VVLVTSSVKEMTERLEDEEEMNAELTAKK | 3309.75 |
| SEQ ID NO: 18 | Amg 27119 3C | VKEMTERLEDEEEMNAELTAKKVVLVTSS | 3309.75 |
| SEQ ID NO: 19 | Amg 27119 4N | CLHRGNSCVKEMTERLEDEEEMNAELTAKK | 3494.93 |
| SEQ ID NO: 20 | Amg 27119 4C | VKEMTERLEDEEEMNAELTAKKCLHRGNSC | 3494.93 |
| SEQ ID NO: 21 | Heart homing | GGGVFWQ | |
| | peptide tag 1 | | |
| SEQ ID NO: 22 | Heart homing | HGRVRPH | |
| | peptide tag 2 | | |
| SEQ ID NO: 23 | Heart homing | VVLVTSS | |
| | peptide tag 3 | | |
| SEQ ID NO: 24 | Heart homing | CLHRGNSC | |
| | peptide tag 4 | | |

As set forth in Table 2, the cardiac-homing peptide sequences (1-4) are tagged at either the N-terminal (XN) or C-terminal end (XC) of the peptide, where ‘X’ corresponds to the reference number of heart homing peptide. For example, Amg 27119 1N comprises the Amg 27119 peptide and cardiac-homing peptide sequence 1 added to the N-terminus thereof, and so forth.

Aspects of the present disclosure can be described with reference to the following numbered clauses, with preferred features laid out in dependent clauses.

A first aspect of the present disclosure is directed to a myopeptide comprising a myosin S2 fragment comprising a sequence having at least 90% sequence identity to an amino acid sequence consisting of VKEMTERLEDEEEMNAELTAKK (SEQ ID NO: 1); and a cardiac-homing peptide tag.

A second aspect of the present disclosure may include the first aspect, wherein the myosin S2 fragment comprises a sequence having at least 95%, at least 96%, at least 97%, at least 98%, or at least 99% sequence identity to SEQ ID NO: 1.

A third aspect of the present disclosure may include any preceding aspect, wherein the myosin S2 fragment comprises a sequence consisting of SEQ ID NO: 1 or SEQ ID NO: 2.

A fourth aspect of the present disclosure may include any preceding aspect. wherein the myopeptide comprises fewer than 50 amino acids.

A fifth aspect of the present disclosure may include any preceding aspects, wherein the myosin S2 fragment comprises fewer than 25 amino acids.

A sixth aspect of the present disclosure may include any preceding aspects, wherein the cardiac-homing peptide tag comprises an amino acid sequence selected from the group consisting of SEQ ID NO: 21, SEQ ID NO: 22, SEQ ID NO: 23, and SEQ ID NO: 24.

A seventh aspect of the present disclosure may include any preceding aspects, wherein the myopeptide is selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

An eighth aspect of the present disclosure may include any preceding aspects, wherein the cardiac-homing peptide tags are added to either the N-terminus, the C-terminus, or both the N-terminus and the C-terminus of SEQ ID NO: 1 or SEQ ID NO: 2.

A ninth aspect of the present disclosure is directed to a pharmaceutical composition comprising the myopeptide according to any of the first through eighth aspects; and one or more pharmaceutically-acceptable excipients.

A tenth aspect of the present disclosure may include the ninth aspect, wherein the pharmaceutical composition is formulated for injection or infusion.

An eleventh aspect of the present disclosure is directed to a method of treating heart failure in a subject in need thereof, the method comprising administering to the subject an effective amount of a myopeptide comprising a myosin S2 fragment comprising a sequence having at least 90% sequence identity to an amino acid sequence consisting of VKEMTERLEDEEEMNAELTAKK (SEQ ID NO: 1).

A twelfth aspect of the present disclosure may include the eleventh aspect, wherein the myosin S2 fragment comprises a sequence having at least 95%, at least 96%, at least 97%, at least 98%, or at least 99% sequence identity to SEQ ID NO: 1.

A thirteenth aspect of the present disclosure may include the eleventh or twelfth aspects. wherein the myosin S2 fragment comprises a sequence consisting of SEQ ID NO: 1 or SEQ ID NO: 2.

A fourteenth aspect of the present disclosure may include any one of the eleventh through thirteenth aspects, wherein the myopeptide further comprises a cardiac-homing peptide tag.

A fifteenth aspect of the present disclosure may include the any one of the eleventh through fourteenth aspects, wherein the cardiac-homing peptide tag comprises an amino acid sequence selected from the group consisting of SEQ ID NO: 21, SEQ ID NO: 22, SEQ ID NO: 23, and SEQ ID NO: 24.

A sixteenth aspect of the present disclosure may include any one of the eleventh through fifteenth aspects, wherein the myopeptide comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

A seventeenth aspect of the present disclosure may include any one of the eleventh through sixteenth aspects, wherein administering comprises injection or infusion.

An eighteenth aspect of the present disclosure may include any one of the eleventh through seventeenth aspects, wherein the myopeptide is administered via injection to the cardiac muscle of the subject.

A nineteenth aspect of the present disclosure may include any one of the eleventh through eighteenth aspects, wherein a maximal force produced by the cardiac muscle of the subject is increased.

A twentieth aspect of the present disclosure may include any one of the eleventh through nineteenth aspects. wherein the maximal force produced by the cardiac muscle of the subject is increased by about 30%.

A twenty-first aspect of the present disclosure may include any one of the eleventh through twentieth aspects, wherein a rate of actin and myosin cross-bridge formation in the cardiac muscle of the subject is increased.

A twenty-second aspect of the present disclosure may include any one of the eleventh through twenty-first aspects, wherein the rate of actin and myosin cross-bridge formation is increased by a factor of about 2.5.

A twenty-third aspect of the present disclosure may include any one of the eleventh through twenty-second aspects, wherein the method does not alter calcium sensitivity of the cardiac muscle of the subject.

A twenty-fourth aspect of the present disclosure may include any one of the eleventh through twenty-third aspects, wherein the subject is suffering from diastolic heart failure with preserved ejection fraction (HFpEF) or systolic heart failure with reduced ejection fraction (HFrEF).

A twenty-fifth aspect of the present disclosure may include any one of the eleventh through twenty-fourth aspects, wherein the S2 myosin fragment binds dephosphorylated cardiac myosin binding protein-C (cMyBP-C).

All documents cited are incorporated herein by reference; the citation of any document is not to be construed as an admission that it is prior art with respect to the present invention.

The foregoing description is illustrative of particular embodiments of the invention but is not meant to be a limitation upon the practice thereof. While particular embodiments have been illustrated and described. it would be obvious to one skilled in the art that various other changes and modifications can be made without departing from the spirit and scope of the invention. It is therefore intended to cover in the appended claims all such changes and modifications that are within the scope of this invention.

1. A myopeptide comprising:
(a) a myosin S2 fragment comprising a sequence having at least 90% sequence identity to an amino acid sequence consisting of VKEMTERLEDEEEMNAELTAKK (SEQ ID NO: 1); and
(b) a cardiac-homing peptide tag.

2. The myopeptide according to claim 1, wherein the myosin S2 fragment comprises a sequence having at least 95%, at least 96%, at least 97%, at least 98%, or at least 99% sequence identity to SEQ ID NO: 1.

3. The myopeptide according to claim 1, wherein the myosin S2 fragment comprises a sequence consisting of SEQ ID NO: 1 or SEQ ID NO: 2.

4. The myopeptide according to claim 1, wherein the myopeptide comprises fewer than 50 amino acids.

5. The myopeptide according to claim 4, wherein the myosin S2 fragment comprises fewer than 25 amino acids.

6. The myopeptide according to claim 1, wherein the cardiac-homing peptide tag comprises an amino acid sequence selected from the group consisting of SEQ ID NO: 21, SEQ ID NO: 22, SEQ ID NO: 23, and SEQ ID NO: 24.

7. The myopeptide according to claim 1, wherein the myopeptide comprises an amino acid sequence selected from the group consisting of SEQ ID NO: 13, SEQ ID NO: 14,SEQ ID NO: 15, SEQ ID NO: 16, SEQ ID NO: 17, SEQ ID NO: 18, SEQ ID NO: 19, and SEQ ID NO: 20.

8. The myopeptide according to claim 3, wherein the cardiac-homing peptide tags are added to the N-terminus, the C-terminus, or both the N-terminus and the C-terminus of SEQ ID NO: 1 or SEQ ID NO: 2.

9. A pharmaceutical composition comprising:
an effective amount of the myopeptide according to claim 1; and
one or more pharmaceutically-acceptable excipients.

10. The pharmaceutical composition according to claim 9, wherein the composition is formulated for injection or infusion.

11. A method of treating heart failure in a subject in need thereof, the method comprising administering to the subject an effective amount of a myopeptide comprising a myosin S2 fragment comprising a sequence having at least 90% sequence identity to an amino acid sequence consisting of VKEMTERLEDEEEMNAELTAKK (SEQ ID NO: 1).

12. The method according to claim 11, wherein the myosin S2 fragment comprises a sequence having at least 95%, at least 96%, at least 97%, at least 98%, or at least 99% sequence identity to SEQ ID NO: 1.

13. The method according to claim 12, wherein the myosin S2 fragment comprises a sequence consisting of SEQ ID NO: 1 or SEQ ID NO: 2.

14. The method according to claim 11, wherein the myopeptide further comprises a cardiac-homing peptide tag.

15. The method according to claim 14, wherein the cardiac-homing peptide tag comprises an amino acid sequence selected from the group consisting of SEQ ID NO: 21, SEQ ID NO: 22, SEQ ID NO: 23, and SEQ ID NO: 24.

16. The method according to claim 11, wherein the myopeptide comprises an amino acid sequence selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

17. The method according to claim 11, wherein administering comprises injection or infusion.

18. The method according to claim 11, wherein the myopeptide is administered via injection to cardiac muscle of the subject.

19. The method according to claim 18, wherein a maximal force produced by the cardiac muscle of the subject is increased.

20. The method according to claim 19, wherein the maximal force produced by the cardiac muscle of the subject is increased by about 30%.

21. The method according to claim 18, wherein a rate of actin and myosin cross- bridge formation in the cardiac muscle of the subject is increased.

22. The method according to claim 21, wherein the rate of actin and myosin cross- bridge formation is increased by a factor of about 2.5.

23. The method according to claim 18, wherein the method does not alter calcium sensitivity of the cardiac muscle of the subject.

24. The method according to claim 11, wherein the subject is suffering from diastolic heart failure with preserved ejection fraction (HFpEF) or systolic heart failure with reduced ejection fraction (HFrEF).

25. The method according to claim 11, wherein the S2 myosin fragment binds dephosphorylated cardiac myosin binding protein-C (cMyBP-C).


SEQUENCE LISTING (24 sequences)

SEQ ID NO: 1 - protein, 22 aa, Mus musculus
VKEMTERLEDEEEMNAELTAKK

SEQ ID NO: 2 - protein, 22 aa, Homo sapiens
VKEMNERLEDEEEMNAELTAKK

SEQ ID NO: 3 - protein, 126 aa, Mus musculus
PLLKSAETEKEMANMKEEFGRVKDALEKSEARRKELEEKMVSLLQEKNDLQLQVQAEQDNLNDAEERCDQLIKNKIQLEAKVKEMTERLEDEEEMNAELTAKKRKLEDECSELKKDIDDLELTLAK

SEQ ID NO: 4 - protein, 129 aa, Homo sapiens
GSSPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAK

SEQ ID NO: 5 - protein, 40 aa, Mus musculus
PLLKSAETEKEMANMKEEFGRVKDALEKSEARRKELEEKM

SEQ ID NO: 6 - protein, 43 aa, Mus musculus
RKELEEKMVSLLQEKNDLQLQVQAEQDNLNDAEERCDQLIKNK

SEQ ID NO: 7 - protein, 36 aa, Mus musculus
CDQLIKNKIQLEAKVKEMTERLEDEEEMNAELTAKK

SEQ ID NO: 8 - protein, 45 aa, Mus musculus
VKEMTERLEDEEEMNAELTAKKRKLEDECSELKKDIDDLELTLAK

SEQ ID NO: 9 - protein, 31 aa, synthetic construct
ETEKEMAMVSLLQAEERCDQDIDDLELTLAK

SEQ ID NO: 10 - protein, 14 aa, Mus musculus
DEEEMNAELTAKKR

SEQ ID NO: 11 - protein, 22 aa, Mus musculus
KLEDECSELKKDIDDLELTLAK

SEQ ID NO: 12 - protein, 15 aa, Mus musculus
ETEKEMAMVSLLQAE

SEQ ID NO: 13 - protein, 29 aa, synthetic construct
GGGVFWQVKEMTERLEDEEEMNAELTAKK

SEQ ID NO: 14 - protein, 29 aa, synthetic construct
VKEMTERLEDEEEMNAELTAKKGGGVFWQ

SEQ ID NO: 15 - protein, 29 aa, synthetic construct
HGRVRPHVKEMTERLEDEEEMNAELTAKK

SEQ ID NO: 16 - protein, 29 aa, synthetic construct
VKEMTERLEDEEEMNAELTAKKHGRVRPH

SEQ ID NO: 17 - protein, 29 aa, synthetic construct
VVLVTSSVKEMTERLEDEEEMNAELTAKK

SEQ ID NO: 18 - protein, 29 aa, synthetic construct
VKEMTERLEDEEEMNAELTAKKVVLVTSS

SEQ ID NO: 19 - protein, 30 aa, synthetic construct
CLHRGNSCVKEMTERLEDEEEMNAELTAKK

SEQ ID NO: 20 - protein, 30 aa, synthetic construct
VKEMTERLEDEEEMNAELTAKKCLHRGNSC

SEQ ID NO: 21 - protein, 7 aa, synthetic construct
GGGVFWQ

SEQ ID NO: 22 - protein, 7 aa, synthetic construct
HGRVRPH

SEQ ID NO: 23 - protein, 7 aa, synthetic construct
VVLVTSS

SEQ ID NO: 24 - protein, 8 aa, synthetic construct
CLHRGNSC